(19) United States
(12) Patent Application Publication
(10) Pub. No.: US 2024/0299183 A1
(43) Pub. Date: Sep. 12, 2024

(54) FUSION CAGE

(71) Applicants: Affiliated Hospital of Youjiang Medical University for Nationalities, Guangxi Zhuang Autonomous Region (CN); Shanghai Jiao Tong University, Shanghai (CN); Shandong University of Science and Technology, Qingdao (CN)

(72) Inventors: Yujin TANG, Guangxi Zhuang Autonomous Region (CN); Jia LIU, Guangxi Zhuang Autonomous Region (CN); Yuting LV, Guangxi Zhuang Autonomous Region (CN); Guohao LIU, Guangxi Zhuang Autonomous Region (CN); Binghao WANG, Guangxi Zhuang Autonomous Region (CN); Liqiang WANG, Guangxi Zhuang Autonomous Region (CN)

(21) Appl. No.: 18/588,549

(22) Filed: Feb. 27, 2024

Related U.S. Application Data
(63) Continuation of application No. 17/648,444, filed on Jan. 20, 2022.

(51) Int. Cl. A61F 2/44

(52) U.S. Cl. CPC A61F 2/4455

(57) ABSTRACT

The present disclosure relates to the technical field of medical supplies, in particular to a fusion cage. The fusion cage comprises a main body, which is a flat-shaped porous structural body and composed of a plurality of structural units. The fusion cage provided by the present disclosure is the flat-shaped porous structural body composed of the plurality of structural units, the porosity of the whole structure of the fusion cage is controlled by using the parametrization design, so the elastic modulus of a fusion body is effectively reduced, and then each structural unit comprises the basal body and the plurality of extension portions extending from the surface of the basal body, the surfaces of the basal body and/or the extension portions are composed of the plurality of curved surfaces through smooth connection, and such design effectively reduces the problem of stress concentration, and improving the postoperative recovery effect of patients.

### CROSS REFERENCE TO RELATED APPLICATION

This application is a continuation of U.S. application Ser. No. 17/648,444, entitled “FUSION CAGE,” filed Jan. 20, 2022, and the entire contents of which are incorporated herein by reference.

### TECHNICAL FIELD

The present disclosure relates to the technical field of medical supplies, in particular to a fusion cage.

### BACKGROUND

The polyether-ether-ketone (PEEK) is researched by French Scient'X Company and has been used for clinical since 1997. As a thermoplastics polymer, PEEK has mechanical properties of high strength, high rigidity, corrosion resistance and hydrolysis resistance as well as better biocompatibility. However, the surface of the PEEK fusion cage has a lower osteogenic efficiency, so scientists have specifically researched a metal fusion cage.

It has found through experimental research that Ti6Al4V has strong fatigue resistance and corrosion resistance compared with other metals. The Ti6Al4V also has better biocompatibility. When titanium is implanted into an animal, the Ti6Al4V has less rejection reaction compared with other metals. With the clinical application, the disadvantages of the existing Ti6Al4V fusion cage come out gradually: due to an unreasonable structure design of the existing Ti6Al4V fusion cage, an elastic modulus is higher (110 GPa), causing unmatched mechanical properties, a lot of stress concentrated in an implant, and sinking and loosening of an adjacent vertebral body after an operation.

### SUMMARY

In order to solve the technical problem, the technical solution adopted by the present disclosure is a fusion cage, comprising a main body, which is a flat-shaped porous structural body and composed of a plurality of structural units, wherein each structural unit comprises a basal body and a plurality of extension portions extending from the surface of the basal body, the surface of the basal body and/or the surfaces of the extension portions are composed of a plurality of curved surfaces through smooth connection, and a porosity of the main body is higher than 40%.

Further, the structures of the basal body and the extension portions are obtained through parametrization design, and the parametrization formula is

$$φ ( x , y , 𝓏 ) = sin ( \frac{2 π}{L} x ) cos ( \frac{2 π}{L} y ) + sin ( \frac{2 π}{L} 𝓏 ) cos ( \frac{2 π}{L} x ) + sin ( \frac{2 π}{L} y ) cos ( \frac{2 π}{L} 𝓏 ) = C ,$$

wherein L is a size parameter of a hole unit, C is a porosity parameter, and variables x, y and z represent structural parameters of three directions in space.

Further, the basal body is spherical, and the plurality of extension portions are uniformly distributed along the periphery of the spherical basal body.

Further, six extension portions are provided, and the axes of two adjacent extension portions are mutually perpendicular to each other.

Further, the structures of the basal body and the extension portions are obtained through parametrization design, and the parametrization formula is

$$φ ( x , y , 𝓏 ) = cos ( \frac{2 π}{L} x ) + cos ( \frac{2 π}{L} y ) + cos ( \frac{2 π}{L} 𝓏 ) = C ,$$

wherein L is the size parameter of the hole unit, C is the porosity parameter, and the variables x, y and z represent the structural parameters of three directions in space.

Further, at least two extension portions are intersected with each other and enclose with the surface of the basal body to form a pore structure.

Further, the structures of the basal body and the extension portions are obtained through parametrization design, and the parametrization formula is:

$$φ ( x , y , 𝓏 ) = 2 [ cos ( \frac{2 π}{L} x ) cos ( \frac{2 π}{L} y ) + cos ( \frac{2 π}{L} 𝓏 ) cos ( \frac{2 π}{L} x ) + \text{} cos ( \frac{2 π}{L} y ) cos ( \frac{2 π}{L} 𝓏 ) ] - [ \text{} cos ( \text{} \frac{4 π}{L} x ) + cos ( \frac{4 π}{L} y ) + cos ( \frac{4}{L} 𝓏 ) ] = C ,$$

wherein L is the size parameter of the hole unit, C is the porosity parameter, and the variables x, y and z represent the structural parameters of three directions in space.

Further, eight extension portions are provided, each extension portion comprises a cylindrical portion and a vertebra portion, the vertebra portion comprises a first end face, a second end face and a third end face, and the first end face, the second end face and the third end face extend internally in an umbrella shape from the side of the cylindrical portion and then are intersected with each other.

Further, the first end face, the second end face and the third end face are mutually perpendicular to each other.

Further, the structural unit is a body-centered cubic structure, the basal body is located in the center of the cubic structure, and the eight extension portions extend to eight corners of the cubic structure.

Further, the extension portions are columnar or tapered.

Further, the structural unit is a diamond structure.

The present disclosure has the following beneficial effects: the fusion cage provided by the present disclosure is the flat-shaped porous structural body composed of the plurality of structural units, the porosity of the whole structure of the fusion cage is controlled by using the parametrization design, so the elastic modulus of a fusion body is effectively reduced, and then each structural unit comprises a basal body and a plurality of extension portions extending from the surface of the basal body, the surfaces of the basal body and/or the extension portions are composed of a plurality of curved surfaces through smooth connection. Such design may effectively reduce the problem of stress concentration, and is beneficial to cell adhesion and improving the postoperative recovery effect of patients.

### BRIEF DESCRIPTION OF THE DRAWINGS

The above and/or additional aspects and advantages of the present disclosure shall be apparent and easy to understand in combination with the description to the embodiments through the accompanying drawings below, wherein:

FIG. 1 is a structural schematic diagram of a structural unit in embodiment I of a fusion cage provided by the present disclosure.

FIG. 2 is a structural schematic diagram of a structural unit in embodiment II of a fusion cage provided by the present disclosure.

FIG. 3 is a structural schematic diagram of a structural unit in embodiment III of a fusion cage provided by the present disclosure.

FIG. 4 is a structural schematic diagram of a structural unit in embodiment IV of a fusion cage provided by the present disclosure.

FIG. 5 is a structural schematic diagram of a structural unit in embodiment V of a fusion cage provided by the present disclosure.

FIG. 6 is a structural schematic diagram of a fusion cage provided by the present disclosure.

FIG. 7 is a schematic diagram for a relation function between a porosity and a porosity parameter C in the present disclosure.

FIG. 8 is a schematic diagram of a modeling flow of a fusion cage provided by the present disclosure.

FIG. 9 is a schematic diagram of a modeling flow of a body-centered cubic structure and a diamond structure in the present disclosure.

### DETAILED DESCRIPTION OF THE EMBODIMENTS

In order to make the purpose, technical solution and advantages of the present disclosure more clear, the present disclosure will be further described in details in combination with the accompanying drawings and embodiments below. It is understood that the embodiments described herein are only used for explaining the present disclosure instead of limiting the present disclosure.

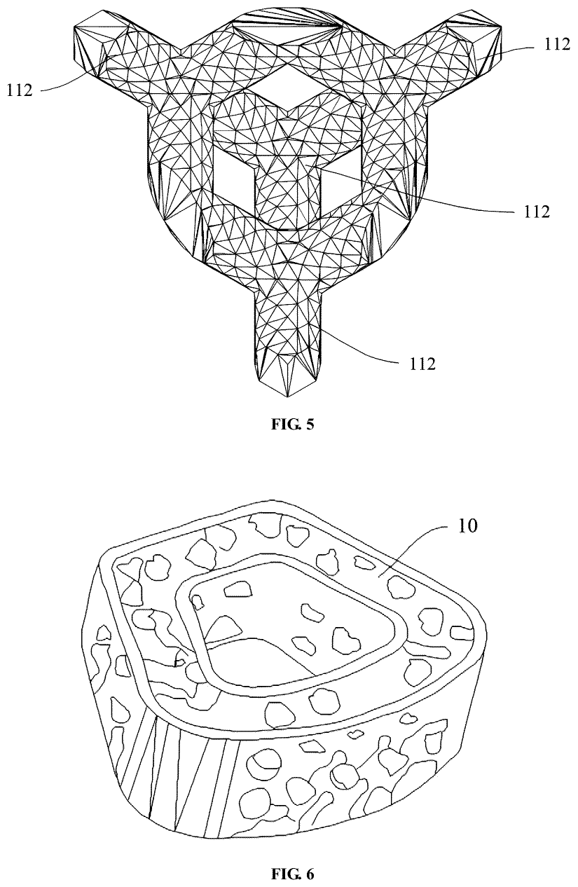

The fusion cage as shown in FIG. 6 is integrally formed by adopting Ti6Al4V in a 3D printing method. The fusion cage comprises a main body 10, which is a flat-shaped porous structural body. A certain angle of inclination is between the upper and lower end faces of the main body 10, the relative angle of inclination of the upper and lower end faces may be confirmed according to the actual situations of patients, and specifically the main body 10 is composed of plurality of structural units. The specific composition method may be: the plurality of structural units are fused through the 3D printing method, and form the flat-shaped porous structural body, and then the fusion cage is formed. Each structural unit comprises a basal body 111 and a plurality of extension portions 112 extending from the surface of the basal body 111, the surface of the basal body 111 and/or the surfaces of the extension portions 112 are composed of a plurality of curved surfaces through smooth connection, so that the surfaces of the basal body 111 and the extension portions 112 are in arc-shaped smooth connection, and then a break angle is avoided. Such design may effectively reduce the elastic modulus of the main body 10 and eliminate the problem of stress concentration. It is noted that the plurality of curved surfaces have tiny area, the surfaces of the basal body 111/extension portions 112 may be regarded as to be formed by dividing the plurality of curved surfaces with tiny area, and the two adjacent curved surfaces are in smooth connection. We have found that the specific surface area and the permeability of the fusion cage may be effectively increased when the porosity of the fusion cage is greater than 40%, the osteogenic property of the fusion cage is further improved, and when the surfaces of the basal body 111 and the extension portions 112 are composed of the curved surfaces through smooth connection, the porosity of the fusion cage is greater than 40%, so that the elastic modulus of the porous structural body matches with the cortical bone and the cancellous bone.

### Embodiment I

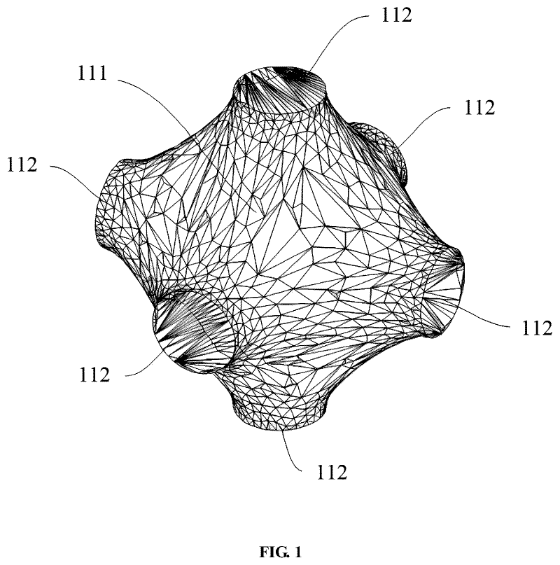

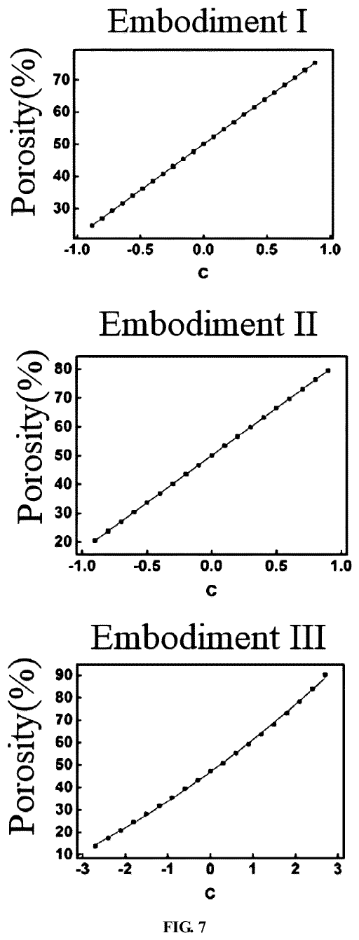

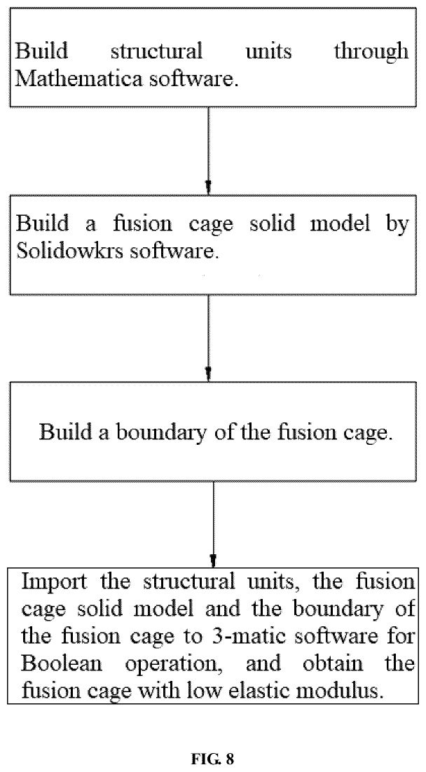

Referring to FIG. 1 and FIG. 7, the whole structure optimization of the structural unit may be obtained through parametrization design, namely, the structures and the relative position relation of the basal body 111 and the extension portions 112 under this embodiment may be obtained through the parametrization design, and the parametrization formula is

$$φ ( x , y , 𝓏 ) = sin ( \frac{2 π}{L} x ) cos ( \frac{2 π}{L} y ) + sin ( \frac{2 π}{L} 𝓏 ) cos ( \frac{2 π}{L} x ) + sin ( \frac{2 π}{L} y ) cos ( \frac{2 π}{L} 𝓏 ) = C ,$$

wherein L is a size parameter of a hole unit, and C is a porosity parameter. For the structural unit modeled and formed through the parametrization formula, the basal body 111 is spherical, a plurality of extension portions 112 are uniformly distributed along the periphery of the spherical basal body 111, the two adjacent structural units are formed by connecting the extension portions 112, and the plurality of structural units are connected so as to form the flat-shaped porous structural body. It is noted that the connection between two extension portions 112 of two different structural units is also the smooth connection, namely, a porous structure in the porous structural body is a smooth curved surface, so as to further eliminate the problem of stress concentration and reduce the elastic modulus. Six extension portions 112 are provided on the basal body 111, the axes of two adjacent extension portions 112 are mutually perpendicular to each other, and the intersection point of a connecting line between two symmetrically arranged extension portions 112 and a connecting line between another two symmetrically arranged extension portions 112 is located at the centre of sphere. At this time, the structural unit is a sphere with symmetrical structures, and six symmetrically arranged extension portions 112 are provided on the outer surface of the sphere and similar to synaptic structures. The plurality of structural units are connected through the mutual fusion of the extension portions 112 so as to form the flat-shaped porous structural body. It is noted that the structures of the basal body 111 and the extension portions 112 are built through formula the parametrization

$$φ ( x , y , 𝓏 ) = sin ( \frac{2 π}{L} x ) cos ( \frac{2 π}{L} y ) + sin ( \frac{2 π}{L} 𝓏 ) cos ( \frac{2 π}{L} x ) + sin ( \frac{2 π}{L} y ) cos ( \frac{2 π}{L} 𝓏 ) = C$$

in the embodiment, the surfaces of the basal body 111 and the extension portions 112 are in smooth connection through the plurality of curved surfaces to form the structural units, and the fusion cage is built by connecting the plurality of structural units mutually. The porosity of the fusion cage may be adjusted by adjusting C value in the parametrization formula, the mechanical property is changed due to the change of the porosity, and then the fusion cage with the porosity and elastic modulus in a suitable scope may be obtained.

### Embodiment II

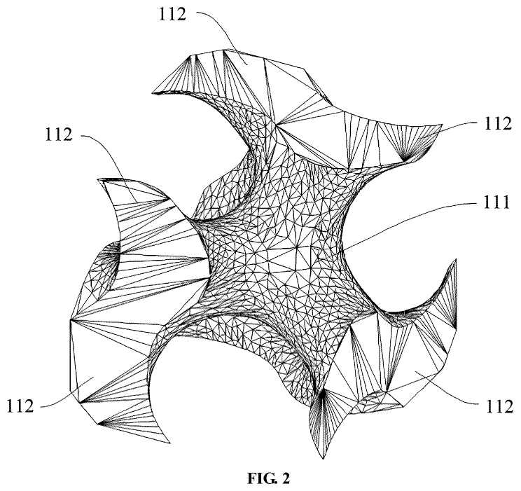

Referring to FIG. 2 and FIG. 7, the whole structure optimization of the structural unit may be obtained through parametrization design, namely, the structures and the relative position relation of the basal body 111 and the extension portions 112 under this embodiment may be obtained through the parametrization design, and the parametrization formula is

$$φ ( x , y , 𝓏 ) = cos ( \frac{2 π}{L} x ) + cos ( \frac{2 π}{L} y ) + cos ( \frac{2 π}{L} 𝓏 ) = C ,$$

wherein L is a size parameter of a hole unit, and C is a porosity parameter. For the structural unit modeled and formed through the parametrization formula, at least two adjacent extension portions 112 on the same basal body 111 are intersected with each other and enclose with the surface of the basal body 111 to form a pore structure, namely, at least one pore structure is on the single structural unit, and when the plurality of structural units are combined to form the main body 10 of the porous structural body, the pore structure may be formed or not be formed between two adjacent structural units. In this embodiment, one face of the pore structure formed by enclosing with the surface of the basal body 111 on the extension portions 112 is composed of a plurality of curved surfaces, and the surface connected to another structural unit may be a plane and may be composed of a plurality of curved surfaces. The surface is in smooth connection with the extension portions 112 of another structural unit through the fusion method. Further, the pore structure in the single structural unit may be adjusted by adjusting the parameters L and C, namely, the porosity and the elastic modulus of the fusion cage may be adjusted according to the actual demand.

### Embodiment III

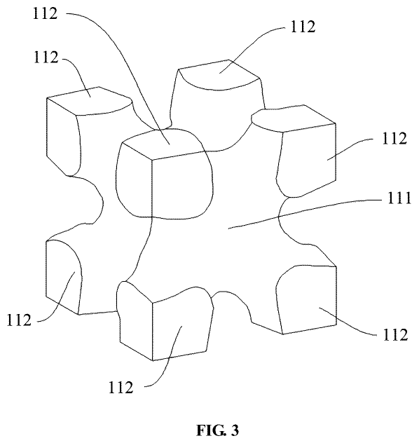

Referring to FIG. 3 and FIG. 7, the whole structure optimization of the structural unit may be obtained through parametrization design, namely, the structures and the relative position relation of the basal body 111 and the extension portions 112 under this embodiment may be obtained through the parametrization design, and the parametrization formula is

$$φ ( x , y , 𝓏 ) = 2 [ cos ( \frac{2 π}{L} x ) + cos ( \frac{2 π}{L} y ) + cos ( \frac{2 π}{L} 𝓏 ) cos ( \frac{2 π}{L} x ) + cos ( \frac{2 π}{L} y ) cos ( \frac{2 π}{L} 𝓏 ) ] - [ \text{} cos ( \frac{4 π}{L} x ) + cos ( \frac{4 π}{L} y ) + cos ( \frac{4 π}{L} 𝓏 ) ] = C ,$$

wherein L is a size parameter of a hole unit, and C is a porosity parameter. For the structural unit modeled and formed through the parametrization formula, eight extension portions 112 are provided and respectively extend outwards from the outer surface of the basal body 111, the eight extension portions 112 are uniformly distributed along the periphery of the sphere, specifically each extension portion 112 comprises a cylindrical portion and a vertebra portion, one end of the cylindrical portion is in smooth connection with the basal body 111 and the other end is in smooth connection with the vertebra portion, the vertebra portion is provided with a first end face, a second end face and a third end face, and the first end face, the second end face and the third end face extend internally in an umbrella shape from the other end of the cylindrical portion and then are intersected with each other. Therefore, it can be seen that the extension portions 112 are respectively connected to another three structural units through the first end face, the second end face and the third end face, so as to form the compact flat-shaped porous structural body. After the extension portions 112 are respectively connected to different structural units through the first end face, the second end face and the third end face, the joint is smooth, so as to ensure that the interior of the porous structure is the curved surface or smooth surface structure, and then the problem of internal stress concentration is eliminated. Further, the first end face, the second end face and the third end face are mutually perpendicular to each other, to increase the connection strength of the adjacent structural units and at the same time avoid the interference between two adjacent structural units.

It is noted that, in the above embodiment I, embodiment II and embodiment III, the variables x, y and z represent the structural parameters of three directions in space.

### Embodiment IV

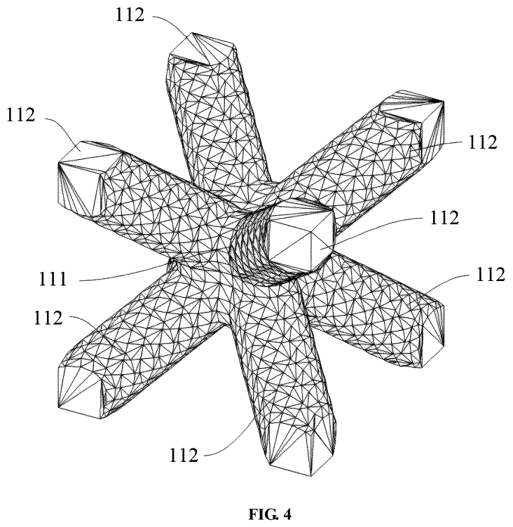

Referring to FIG. 4, in this embodiment, the structural unit is a body-centered cubic structure, the basal body 111 is located in the center of the cubic structure, eight extension portions 112 extend to the eight corners of the cubic structure, and the structural unit is a lattice structure as a whole. At this time, the extension portions 112 may be columnar or tapered, and one end, away from the basal body 111, on the columnar or tapered extension portions 112 is provided with three end faces. Any two end faces of the three end faces are connected and form a conical tip shape, at this time, the extension portions 112 are respectively connected to three extension portions 112 of another three structural units through the three end faces, and a plurality of structural units with the shape are connected so as to form the flat-shaped porous structural body. Researches show that the elastic modulus of the porous structural body fusion cage composed of the structural unit with the body-centered cubic structure is equivalent to the spine cortical bone when the porosity is 50% or above.

### Embodiment V

Referring to FIG. 5, in this embodiment, the structural unit is a diamond structure, at this time a pore structure is at the center of the basal body 111, the extension portions 112 extend from the corner of the basal body 111, and the structural unit is a lattice structure as a whole. One end, away from the basal body 111, of the extension portions 112 is provided with three end faces. Any two end faces of the three end faces are connected and form a conical tip shape, at this time, the extension portions 112 are respectively connected to three extension portions 112 of another three structural units through the three end faces, and a plurality of structural units with the shape are connected so as to form the flat-shaped porous structural body. Researches show that the elastic modulus of the porous structural body fusion cage composed of the structural unit with the body-centered cubic structure is equivalent to the spine cortical bone when the porosity is 60% or above.

It is noted that the structural unit should be established in Mathematica software by using the function formula in embodiment I, embodiment II and embodiment III when the fusion cage is built through the parametrization design structural unit in embodiment I, embodiment II and embodiment III. The porosity change of the structural unit is regulated by controlling the functions L and C, and then a fusion cage solid model with a certain size and a certain angle between the upper and lower end faces is built through Solidowkrs software. Various sizes of the fusion cage solid model and the relative angle of inclination of the upper and lower end faces are adjusted according to the actual situations of patients. Then, a boundary of the fusion cage needs to be built, and finally the structural unit, the fusion cage solid model and the fusion cage boundary are imported to 3-matic software for Boolean operation, namely, the fusion cage with low elastic modulus may be obtained, as shown in FIG. 8.

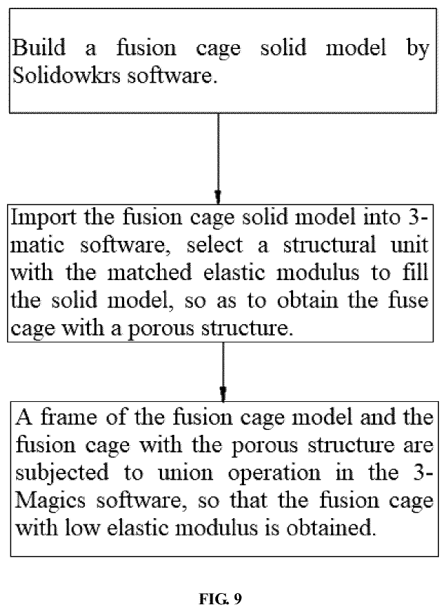

It is noted that the fusion cage solid model with a certain size and a certain angle between the upper and lower end faces is built through the Solidowkrs software when the fusion cage is built through the body-centered cubic structure and the diamond structure, and various sizes of the fusion cage solid model and the relative angle of inclination of the upper and lower end faces are adjusted according to the actual situations of patients. After modeling is completed, the fusion cage solid model is imported to the 3-matic software, the structural unit with the matched elastic modulus, such as the body-centered cubic structure and the diamond structure, is selected to fill the solid model, so that the fusion cage with the porous structure is obtained. The frame of the fusion cage model and the fusion cage with the porous structure are subjected to union operation in the 3-Magics software, so that the fusion cage with low elastic modulus may be obtained, as shown in FIG. 9.

The optional implementations of the present disclosure are described above. It should be noted that those of ordinary skill in the art may further make some improvements and refinements without departing from the principles described in the present disclosure and these improvements and refinements shall also fall within the protection scope of the present disclosure.

What is claimed is:

1. A fusion cage, wherein the fusion cage comprises:
a main body (10), which is a flat-shaped porous structural body and composed of a plurality of structural units,
wherein each structural unit comprises a basal body (111) and a plurality of extension portions (112) extending from the surface of the basal body (111), the surface of the basal body (111) and/or the surfaces of the extension portions (112) are composed of a plurality of curved surfaces through smooth connection,
and a porosity of the main body (10) is higher than 40%.

2. The fusion cage according to claim 1, wherein the structures of the basal body (111) and the extension portions (112) are obtained through parametrization design, and the parametrization formula is:
$$φ ( x , y , 𝓏 ) = sin ( \frac{2 π}{L} x ) cos ( \frac{2 π}{L} y ) + sin ( \frac{2 π}{L} 𝓏 ) cos ( \frac{2 π}{L} x ) + sin ( \frac{2 π}{L} y ) cos ( \frac{2 π}{L} 𝓏 ) = C ,$$
wherein L is a size parameter of a hole unit, C is a porosity parameter, and variables x, y and z represent structural parameters of three directions in space.

3. The fusion cage according to claim 1, wherein the basal body (111) is spherical, and the plurality of extension portions (112) are uniformly distributed along the periphery of the spherical basal body (111).

4. The fusion cage according to claim 3, wherein six extension portions (112) are provided, and the axes of two adjacent extension portions (112) are mutually perpendicular to each other.

5. The fusion cage according to claim 1, wherein the structures of the basal body (111) and the extension portions (112) are obtained through parametrization design, and the parametrization formula is:
$$φ ( x , y , 𝓏 ) = cos ( \frac{2 π}{L} x ) + cos ( \frac{2 π}{L} y ) + cos ( \frac{2 π}{L} 𝓏 ) = C ,$$
wherein L is the size parameter of the hole unit, C is the porosity parameter, and the variables x, y and z represent the structural parameters of three directions in space.

6. The fusion cage according to claim 1, wherein at least two extension portions (112) are intersected with each other and enclose with the surface of the basal body (111) to form a pore structure.

7. The fusion cage according to claim 1, wherein the structures of the basal body (111) and the extension portions (112) are obtained through parametrization design, and the parametrization formula is:
$$φ ( x , y , 𝓏 ) = 2 [ cos ( \frac{2 π}{L} x ) cos ( \frac{2 π}{L} y ) + cos ( \frac{2 π}{L} 𝓏 ) cos ( \frac{2 π}{L} x ) + cos ( \frac{2 π}{L} y ) cos ( \frac{2 π}{L} 𝓏 ) ] - [ \text{} cos ( \frac{2 π}{L} x ) ) + cos ( \frac{2 π}{L} y ) + cos ( \frac{2 π}{L} 𝓏 ) ] = C ,$$
wherein L is the size parameter of the hole unit, C is the porosity parameter, and the variables x, y and z represent the structural parameters of three directions in space.

8. The fusion cage according to claim 1, wherein eight extension portions (112) are provided, each extension portion (112) comprises a cylindrical portion and a vertebra portion, the vertebra portion comprises a first end face, a second end face and a third end face, and the first end face, the second end face and the third end face extend internally in an umbrella shape from the side of the cylindrical portion and then are intersected with each other.

9. The fusion cage according to claim 8, wherein the first end face, the second end face and the third end face are mutually perpendicular to each other.

10. The fusion cage according to claim 1, wherein the structural unit is a body-centered cubic structure, the basal body (111) is located in the center of the cubic structure, and the eight extension portions (112) extend to eight corners of the cubic structure.

11. The fusion cage according to claim 10, wherein the extension portions (112) are columnar or tapered.

12. The fusion cage according to claim 1, wherein the structural unit is a diamond structure.

13. The fusion cage according to claim 2, wherein the basal body (111) is spherical, and the plurality of extension portions (112) are uniformly distributed along the periphery of the spherical basal body (111).

14. The fusion cage according to claim 5, wherein at least two extension portions (112) are intersected with each other and enclose with the surface of the basal body (111) to form a pore structure.

15. The fusion cage according to claim 7, wherein eight extension portions (112) are provided, each extension portion (112) comprises a cylindrical portion and a vertebra portion, the vertebra portion comprises a first end face, a second end face and a third end face, and the first end face, the second end face and the third end face extend internally in an umbrella shape from the side of the cylindrical portion and then are intersected with each other.
